(19) United States
(12) Patent Application Publication
(10) Pub. No.: US 2010/0157240 A1
(43) Pub. Date: Jun. 24, 2010

(54) CORRECTION OF PERIPHERAL DEFOCUS OF AN EYE AND CONTROL OF REFRACTIVE ERROR DEVELOPMENT

(76) Inventors: GREGOR F. SCHMID, CANTON, GA (US); RICK EDWARD PAYOR, CUMMING, GA (US); ALDO A. MARTINEZ, SINGAPORE (SG)

Correspondence Address: CIBA VISION CORPORATION;PATENT DEPARTMENT, 11460 JOHNS CREEK PARKWAY, DULUTH, GA, 30097-1556, US

(21) Appl. No.: 12/639,101

(22) Filed: Dec. 16, 2009

Related U.S. Application Data
(60) Provisional application No. 61/139,051, filed on Dec. 19, 2008.

(51) Int. Cl. G02C 7/04; G02C 7/06; A61B 3/00

(52) U.S. Cl. 351/161; 351/168; 351/246

(57) ABSTRACT

An ophthalmic lens series for reducing the progression of myopia through adequately correcting the peripheral retina, the series comprising more than one ophthalmic lens forming a series. Each ophthalmic lens of the series has a central power level common to the series. Each of the ophthalmic lenses of the series has one differential (peripheral minus central) power level selected from a variety of differential power levels. Providing a variety of differential power levels reduces the risk of over or under-correcting the peripheral retina of a particular eye.

This application claims the benefits under 35 U.S.C. 119(e) of the U.S. Provisional Patent Application No. 61/139,051 filed on Dec. 19, 2008, incorporated herein by reference in its entirety.

### TECHNICAL FIELD

The present invention relates generally to the field of ophthalmic devices. More specifically, the present invention relates to the field of ophthalmic devices for the correction of peripheral defocus of an eye and control of refractive error development.

### BACKGROUND OF THE INVENTION

The myopic (nearsighted) eye has been described anatomically as more elongated axially than at the equator making it less spherical than the emmetropic eye. More recently MRI imaging has confirmed these findings as living human eyes as described by David Atchinson in “Eye Shape in Emmetropia and Myopia” and Krish Singh in “Three Dimensional Modeling of the Human Eye Based on Magnetic Resonance Imaging”. Investigators have found by auto-refraction that there is also a difference in the differential peripheral refractions between the hyperopic, emmetropic and myopic eyes. Such a study is exemplified by Donald Mutti in “Peripheral Refraction and Ocular Shape in Children”.

In these cases the differential peripheral defocus is the change in central to peripheral refraction as a function of the central (normal) refraction used to determine the clinical amount of myopia. In the case where peripheral refraction is more positive (less convergent) and focuses the image further outside or behind the retina than the central refraction, the differential peripheral defocus is said to be hyperopic. Conversely, where differential peripheral refraction is more negative (more convergent) and focuses the image further inside or in front the retina than the central refraction, the differential peripheral defocus is said to be myopic.

Investigators such as Mutti have found by auto-refraction that the differential peripheral defocus is more myopic for eyes with a central hyperopic refraction and more hyperopic for myopic eyes. Myopic defocus comes from light rays passing through the lens that are more convergent and thus come to a focus in front of the retina. This is similar to the uncorrected myopic eye where the axial length of the eye, and more precisely the position of the retina, exceeds the focal length of the optical power of the eye. Myopic defocus is thus said to be light focused inside or in front of the retina. The reverse description applies to hyperopic defocus. Hyperopic defocus comes from light rays passing through the optics of the eye that are less convergent and thus come to a focus outside or behind the retina.

Ophthalmic lenses, including soft contact lenses, comprise a central sphero-cylindrical power which is located at the central axis (or zero axis) on a lens. The central sphero-cylindrical power is the normal specification of an ophthalmic lens used for vision correction based on the subjective refraction to optimize central visual acuity. Ophthalmic lenses additionally comprise a peripheral power profile, which shows the peripheral power values located at a determined distance from the central axis. Previously the peripheral power profile of ophthalmic lenses was left the same or adjusted to reduce spectacle distortion or improve central vision. Due to the lower visual acuity of the peripheral retina, correcting the peripheral refraction was not seen as significant improvement.

Myopic eyes typically exhibit more elongated, prolate shapes than emmetropic eyes. Due to the increasingly prolate shape of the eye ball with increasing myopia, the peripheral retina experiences increasing hyperopic defocus. However, considerable individual variability in differential (peripheral power level minus central power level) refraction was observed in both children and adults of comparable central refractive status. As a consequence, the use of an anti-myopia ophthalmic/contact lens with an average, single, differential lens power would overcorrect the peripheral retina in some myopes, but undercorrect the peripheral retina in other myopes, depending on the individual peripheral defocus of a particular eye.

The optical effect for severe overcorrection of the peripheral retina may be an excessive amount of myopic, peripheral defocus, which not only could hamper peripheral vision but also cause peripheral form vision deprivation resulting in further axial eye growth and myopia progression. The optical effect for under-correction may be a residual amount of hyperopic defocus in the peripheral retina, which would also create a stimulus for axial eye growth and worsening myopia. Using an anti-myopia contact lens with an above-average, single, differential lens power such that in most progressing myopes peripheral hyperopia is converted to peripheral myopia would prevent under-correction in some myopes, but create severe over-correction in other myopes with the above-mentioned consequences.

### SUMMARY OF THE INVENTION

In example embodiments, the present invention provides an ophthalmic lens series for reducing the progression of myopia, the series comprising a plurality (more than one) of ophthalmic lenses. The lens series corrects a peripheral defocus of an eye, and each lens of the ophthalmic lens series has a central power level common to the series. Each of the ophthalmic lenses of the series has one differential lens power level selected from a variety of differential power levels (peripheral power level minus central power level). Providing a variety of peripheral power levels reduces the risk of over-correcting or under-correcting the peripheral defocus of a particular eye.

In an alternative embodiment, the variety of differential lens power levels are selected from a group consisting of: high differential lens power, medium differential lens power, and low differential lens power. In a further alternative embodiment, the lenses in the ophthalmic lens series have a central to peripheral lens power differential range between approximately 0.25 diopter and approximately 4 diopters. In still further embodiments, the lenses in the ophthalmic series may have a negative differential lens power range (i.e., the peripheral lens power levels provided may be more negative than the central power level). The lenses may be made of or comprise soft contact lens material.

In another aspect, the invention is a method for adequately correcting the peripheral defocus of a myopic eye, the method comprising providing a series of ophthalmic lenses, wherein each lens in the series of ophthalmic lenses has a common central power and each lens in the series has one differential lens power level selected from a variety of differential lens powers. The method further comprises selecting a first ophthalmic lens from the series of ophthalmic lenses and placing the first lens on an eye, and then evaluating visual performance of the eye having the first lens, wherein the evaluation determines overcorrection or undercorrection of the peripheral retina. The method further comprises replacing, on the eye, the first lens with an alternative lens from the series having a higher differential lens power for an eye determined to be undercorrected by the first lens or a lens having a lower differential lens power for an eye determined to be overcorrected by the first lens.

In aspects, the variety of differential lens power levels may be selected from a group consisting of high differential lens power, medium differential lens power, and low differential lens power, and the differential lens power range may be between approximately 0.25 diopter and approximately 4 diopters. In further embodiments, the lenses in the ophthalmic series may have a negative differential lens power range (i.e., the peripheral lens power levels provided may be more negative than the central power level). The lenses may be made of or comprise soft contact lens material.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 is a representation of test results for peripheral differential (peripheral minus central) vs. central sphere correction in children at fifteen degrees off-axis as measured during cycloplegia with an open-field autorefractometer using off-axis fixation targets.

FIG. 2 is a representation of test results for peripheral differential (peripheral minus central) vs. central sphere correction in adults at twenty degrees off-axis as measured during cycloplegia with an open-field autorefractometer using off-axis fixation targets.

FIG. 3A is a representation of the effect on peripheral refraction of a lens with a large peripheral power differential as compared to a control lens with uniform power in a subject with about 6 diopters of central myopia.

FIG. 3B is a representation of the effect on peripheral refraction of a lens with a large peripheral power differential as compared to a control lens with uniform power in a subject with about 1.5 diopters of central myopia.

FIG. 4A is a representation of the effect on peripheral refraction of a lens with a small peripheral power differential as compared to a control lens with uniform power in a subject with about 6 diopters of central myopia.

FIG. 4B is a representation of the effect on peripheral refraction of a lens with a small peripheral power differential as compared to a control lens with uniform power in a subject with about 1.5 diopters of central myopia.

FIG. 5 is a representation of the effect of peripheral refraction in terms of sphere refraction and sphere equivalent on rated side vision quality.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

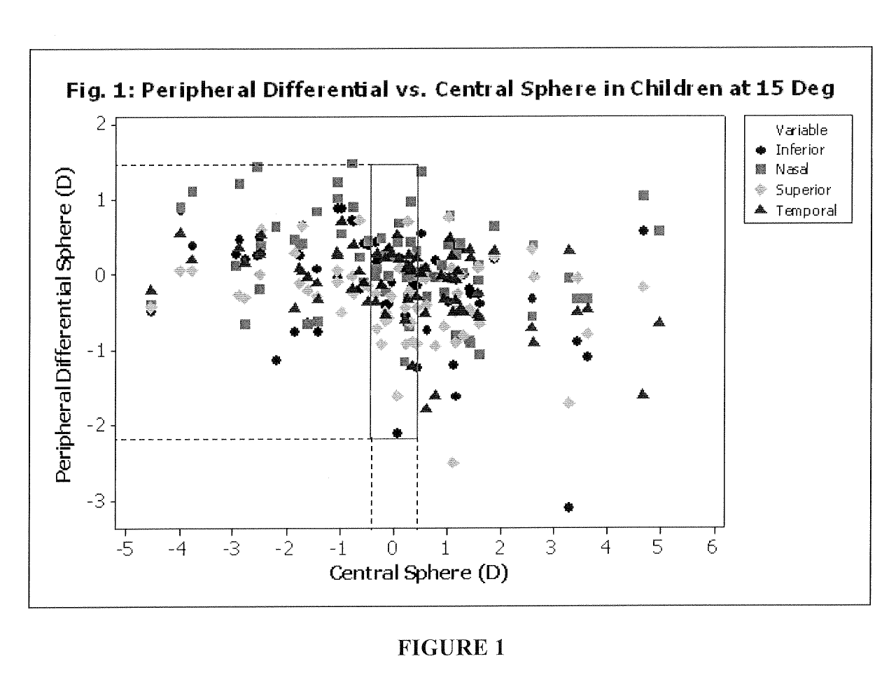

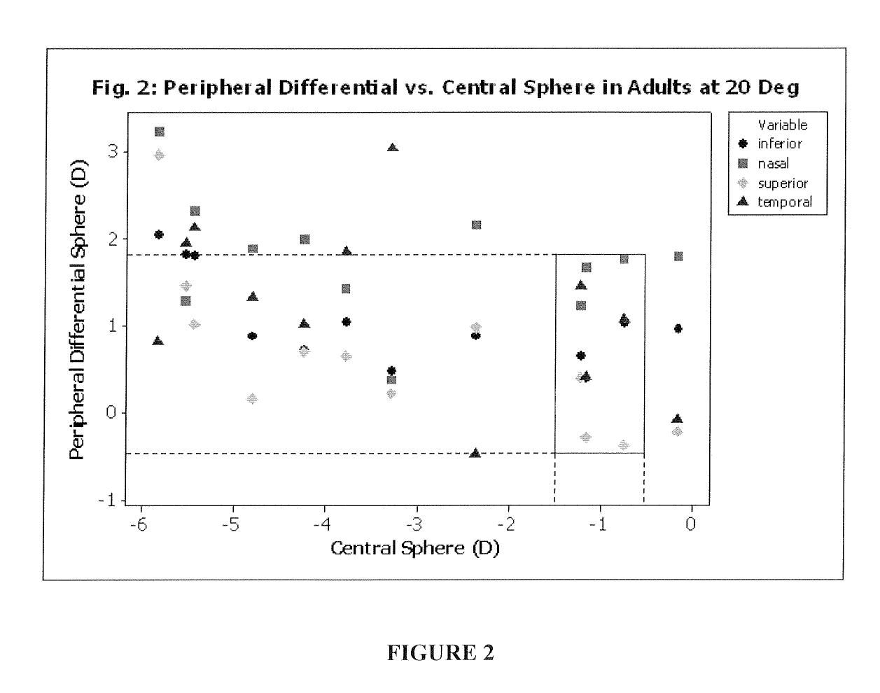

In order to create the desired anti-myopia imagery in any given eye, anti-myopia contact lenses can be provided in a variety of differential (peripheral minus central) lens powers for each center (distance correction) power. In a study of 63 children ages 7-15 years, where refraction was measured with a “Shin-Nippon” K5001 open-field autorefractometer on-axis and off-axis at 15 degrees in right eyes during cycloplegia, it was found that a required peripheral differential lens power (peripheral sphere power minus central sphere power) varied greatly for any central sphere power, i.e. for any refractive status (FIG. 1). Within plus and minus half a diopter of 0.00D central sphere power, for example (box outline), differential lens power ranged from approximately −2.20D to +1.40D. The range was comparable to that around other central sphere powers. A study in both eyes of 6 young adult volunteers revealed a substantial individual variability in differential refraction as well (FIG. 2). Refraction was measured on-axis and off-axis at approximately 20 degrees in both eyes during cycloplegia. Around approximately −1.00D of central sphere, for example (box outline), differential lens power ranged from approximately −0.50D to +1.80D.

These findings demonstrate that anti-myopia lenses having a variety of differential lens powers can avoid undercorrection or severe overcorrection of the peripheral retina in any given eye and produce the desired anti-myopia imagery for many central (distance correction) powers. The efficacy of an example embodiment at various peripheral power levels is further supported by on- and off-axis measurements of refraction with a Welch-Allyn SureSight handheld autorefractometer in adult volunteers at the CIBA Vision Research Clinic.

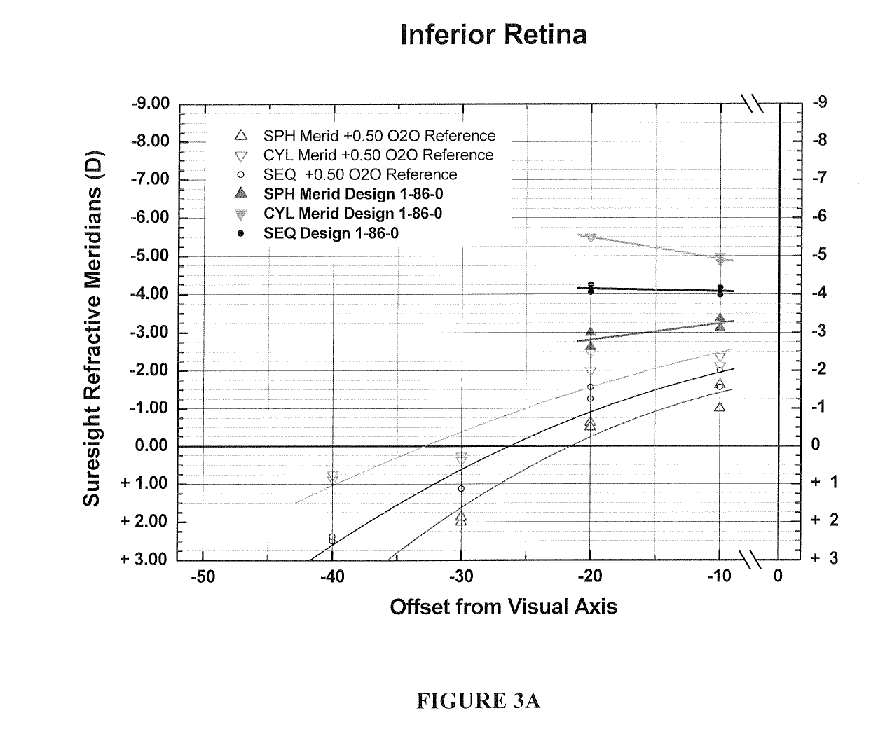

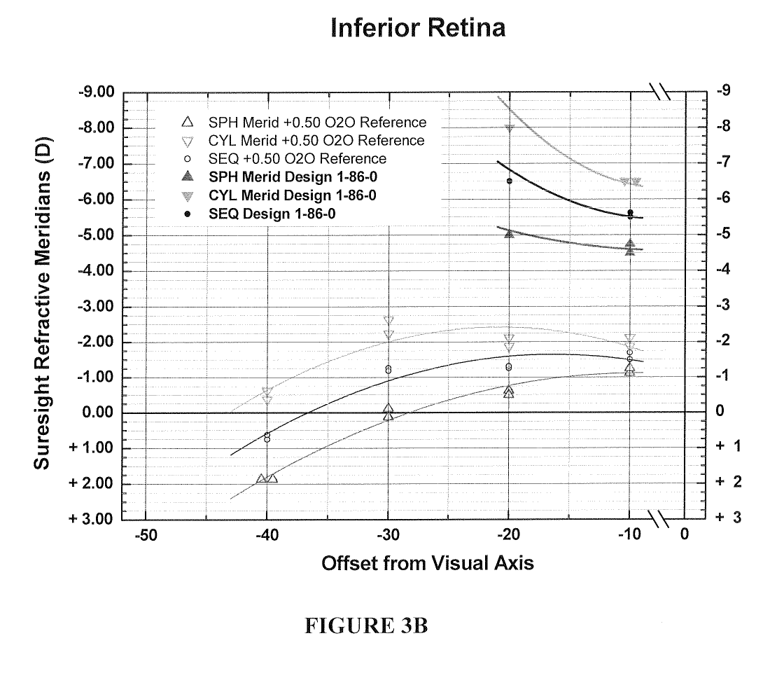

A first Example represented an anti-myopia lens design with a higher amount of differential lens power adequately that corrects larger differential peripheral defocus in subject RP (FIG. 3A), but greatly overcorrects smaller differential peripheral defocus in subject GS (FIG. 3B).

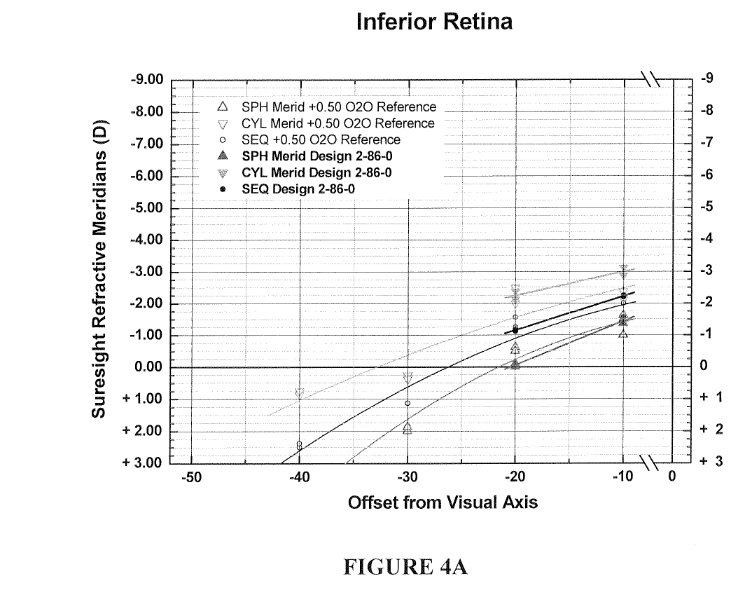

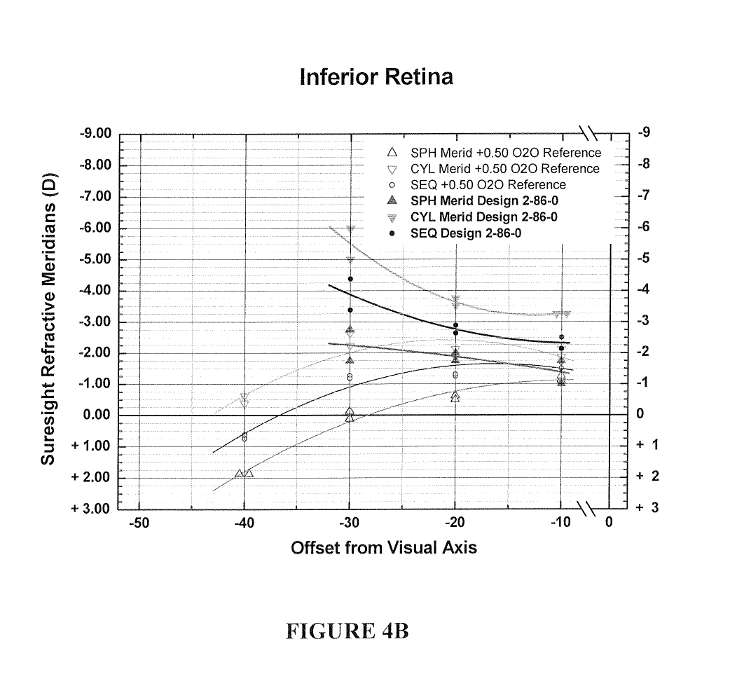

A second Example represented an anti-myopia lens design with less differential lens power, on the other hand, that has little effect on differential peripheral defocus in subject RP (FIG. 4A), but slightly over-corrects the differential peripheral defocus in subject GS (FIG. 4B).

Optical designs of soft contact lenses with positive differential lens powers were shown to adequately correct the peripheral retina for high (2.50D; hyperopic) differential refraction/power. However, the same design worn on an eye requiring lesser amounts of differential lens power over-corrected the peripheral retina, creating severe peripheral myopia and noticeable peripheral blur for the wearer.

A preferred number of differential lens power levels for a given central (distance) power depends on the range of differential refraction within a population, the tolerance to peripheral blur and the accuracy of the mechanism that drives visually guided eye growth. Because it is not a requirement that the contact lens accurately corrects the periphery by focusing an image precisely on the retina, but just moves the spherical line image to the front of—and near—the retina, three different peripheral power levels in a series (e.g., high, medium, low) per center power can be sufficient.

In an example lens series according to the present invention, differential lens powers that are contemplated to custom-correct the various differential defocus can range from approximately +0.25D to +4.00D at thirty degrees off-axis, or more preferably from approximately +1.00D to +3.00D and the high, medium and low differential lens powers may be set at approximately +3.00, +2.00 and +1.00D, respectively.

A method according to the present invention provides selecting ‘high’, ‘medium’ or ‘low’ differential lens powers in clinical practice without advanced knowledge of the individual patient's peripheral refraction. By starting with the ‘high’ differential lens power and evaluating the visual performance, the patient not accepting the lens due to peripheral over-correction will be apparent and indicate moving to the next lower ‘medium’ differential lens power. This can be repeated once more if the ‘low’ differential lens power is required. As an alternative embodiment of the method according to the present invention, starting with the “low” differential lens power and evaluating the visual performance, the patient not accepting the lens due to peripheral under-correction will be apparent and indicate moving to the next higher ‘medium’ differential lens power. This can be repeated once more if the ‘high’ differential lens power is required. By targeting the ‘medium’ differential lens power at the median required differential lens power for a given sphere power (refractive status), the step between the next higher or lower will be determined by the range of clinical tolerance to over-correction of the peripheral refractive error.

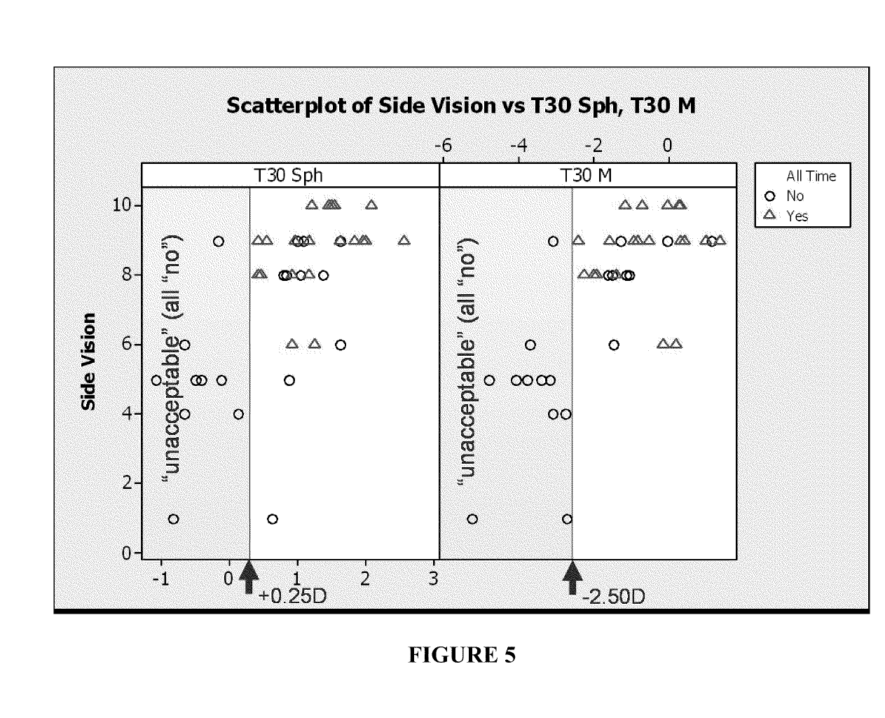

Correlation analysis between subjective vision quality and objective auto-refraction in the retinal periphery of patients who reported differences in vision quality between lenses of various differential lens powers revealed that over-correction limits exist, beyond which vision quality is not acceptable. Turning to FIG. 5, there is shown a representation of the effect of peripheral refraction on the rating of side vision quality for the lenses, using a scale from 0-10. Symbols indicate those patients subjects who answered “no” (circles) or “yes” (triangles) to the question whether vision quality is sufficient to wear the lens all the time.

The plot as shown in FIG. 5 is in terms of sphere refraction (Sph; left side of plot) and sphere equivalent refraction (M; right side of plot) as measured at 30 degrees in the temporal retina (nasal field) (“T30”) by auto-refractometry. If, for example at 30 degrees in the temporal retina (nasal field), the lens produces a sphere refraction below about +0.25D (i.e. on the retina or in front of the retina), then vision quality is unacceptable as indicated by all patients answering “no” to the question whether vision quality is sufficient to wear the lens all the time. This is shown in the plot in the shaded left side of the “T30 Sph” portion. Similarly, for a sphere equivalent refraction below about −2.50D (i.e. further in front of the retina than −2.50D), vision quality is unacceptable as indicated by all patients answering “no” to the question whether vision quality is sufficient to wear the lens all the time (shaded left side of the “T30 M” portion.). The correlation analysis also indicated that lens rejection is chiefly caused by decreased peripheral vision as opposed to central vision. The identification and application of these over-correction limits substantially facilitates the lens fitting procedure, and helps reduce vision degradation and lens rejection by the patient when correcting peripheral defocus and controlling refractive error development.

In an alternative embodiment, a contact lens can be designed with a negative power differential to provide hyperopic defocus in the central and retinal periphery for the stimulation of axial eye growth in hyperopic eyes.

In a further alternative embodiment, a contact lens according to the present invention comprises sphero-cylindrical central power for correcting astigmatism. In this case, either the sphere part or the spherical equivalent (sphere+half of the cylinder) of the central power can be used as central sphere power for defining differential lens power.

Example lenses in the lens series can be composed of any suitable known contact lens materials. Particular examples include soft lens materials, such as hydrogels and silicon hydrogel materials.

While the invention has been described with reference to preferred and example embodiments, it will be understood by those skilled in the art that a variety of modifications, additions and deletions are within the scope of the invention, as defined by the following claims.

What is claimed is:

1. An ophthalmic lens series for correcting a peripheral defocus of an eye, said series comprising:
a plurality of ophthalmic lenses forming a series;
each ophthalmic lens of the series having a central power level common to the series; and
each said ophthalmic lens of said series having one differential lens power level selected from a variety of differential lens power levels;
whereby a lens can be selected from the series to reduce the risk of over-correcting or under-correcting the peripheral defocus of a particular eye.

2. The ophthalmic lens series of claim 1, wherein the variety of differential lens power levels are selected from a group consisting of high differential lens power, medium differential lens power, and low differential lens power.

3. The ophthalmic lens series of claim 1, wherein the lenses in the ophthalmic lens series have a differential lens power range between approximately 0.25 diopter and approximately 4 diopters.

4. The ophthalmic lens series of claim 1, wherein the lenses in the ophthalmic lens series have a negative differential lens power range.

5. The ophthalmic lens series of claim 1, wherein each said lens comprises soft lens material.

6. A method for correcting a peripheral defocus of an eye comprising:
providing a series of ophthalmic lenses, wherein each lens in the series of ophthalmic lenses has a common central power and each lens in the series has one differential lens power level selected from a variety of differential lens power levels;
selecting a first ophthalmic lens from the series of ophthalmic lenses and placing the first lens on an eye;
evaluating visual performance of the eye having the first lens, wherein the evaluation determines overcorrection or undercorrection of the peripheral retina; and
replacing, on the eye, the first lens with an alternative lens from the series having a higher differential lens power for an eye determined to be undercorrected by the first lens or a lens having a lower differential lens power for an eye determined to be overcorrected by the first lens.

7. The method of claim 6, wherein the variety of differential lens power levels are selected from a group consisting of: high differential lens power, medium differential lens power, and low differential lens power.

8. The method of claim 6, wherein the lenses in the ophthalmic lens series have a differential lens power range between approximately 0.25 diopter and approximately 4 diopters.

9. The method of claim 6, wherein the lenses in the ophthalmic lens series have a negative differential lens power range.

10. The method of claim 6, wherein each said lens comprises soft lens material.
